(19) United States
(12) Patent Application Publication
(10) Pub. No.: US 2011/0091604 A1
(43) Pub. Date: Apr. 21, 2011

(54) SYNTHETIC MEAT

(76) Inventor: SETH ADRIAN MILLER, Englewood, CO (US)

(21) Appl. No.: 12/603,459

(22) Filed: Oct. 21, 2009

(51) Int. Cl. A23L 1/314; C12M 1/36

(52) U.S. Cl. 426/56; 426/574; 435/286.1

(57) ABSTRACT

Examples of methods, systems and computer accessible mediums related to producing synthetic meat are generally described herein. In some example methods, a substrate configured to support cell growth may be provided. The substrate may be seeded with cells. The seeded substrate may be rolled through a bioreactor having a roll-to-roll mechanism, thereby allowing nutrients and growth factors to interact with the cells. The seeded substrate may be stretched to simulate muscle action. The seeded substrate may be monitored for uniformity of cell growth as it is rolled through the bioreactor. A film of synthetic meat is obtained from the substrate.

### BACKGROUND

A meat analogue, also called a meat substitute, mock meat, faux meat, or imitation meat, approximates aesthetic qualities (such as texture, flavor, and appearance) and/or chemical characteristics of certain types of meat. For the purposes of discussion, meat analogue, meat substitute, mock meat, and like types of meat will be referred to herein as synthetic meat.

Generally, synthetic meat is understood to mean a food made from non-meats, sometimes without dairy products. The market for synthetic meat includes vegetarians, vegans, non-vegetarians seeking to reduce their meat consumption for health or ethical reasons, and people following religious dietary laws.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are, therefore, not to be considered limiting in scope, the disclosure will be described with additional specificity and detail through the use of the accompanying drawings, in which:

FIG. 1 is a flow diagram of an example method for producing synthetic meat;

FIG. 2 is a flow diagram of an alternative example method of producing synthetic meat;

FIG. 3 is a block diagram of an example system configured to be suitable for producing synthetic meat;

FIG. 4 is a diagram of a suitable bioreactor for use with systems for producing synthetic meat;

FIG. 5 is a diagram of a bioreactor such as shown in FIG. 3 in a stacked configuration;

FIG. 6 is a block diagram of a computing device that may be used in producing synthetic meat according to certain examples; and

FIG. 7 illustrates a block diagram of an example computer program product; all arranged in accordance with at least some examples of the present disclosure.

### DETAILED DESCRIPTION

In the following detailed description, reference is made to the accompanying drawings, which form a part hereof. In the drawings, similar symbols typically identify similar components, unless context dictates otherwise. The illustrative examples described in the detailed description, drawings, and claims are not meant to be limiting. Other examples may be utilized, and other changes may be made, without departing from the spirit or scope of the subject matter presented herein. It will be readily understood that the aspects of the present disclosure, as generally described herein, and illustrated in the figures, may be arranged, substituted, combined, separated, and designed in a wide variety of different configurations, all of which are explicitly contemplated herein.

The present disclosure is generally drawn to methods, systems, apparatuses, and computer programs related to producing synthetic meat. In one example, a method generally comprises growing synthetic meat in a continuous process on long, wide sheets of porous, degradable polymer support. This method may be used to grow films of synthetic meat at a thickness of 0.5 mm or less such that vascularization may be omitted. Meats produced using the methods described herein may be used, for example, for deli meats.

The field of synthetic meat is a relatively young, and is still seeking improvements in its technical capabilities and its approach to the market. The biggest opportunities for synthetic meat are generally seen in the arena of processed foods, such as hamburger replacement or chicken nuggets. However, few viable approaches towards making meats for these uses have been suggested, especially at a manufacturing scale.

In accordance with the present disclosure, various examples methods for producing synthetic meat may comprise providing a substrate (also referred to as a support structure), seeding the substrate, and growing meat from the seeding of the substrate.

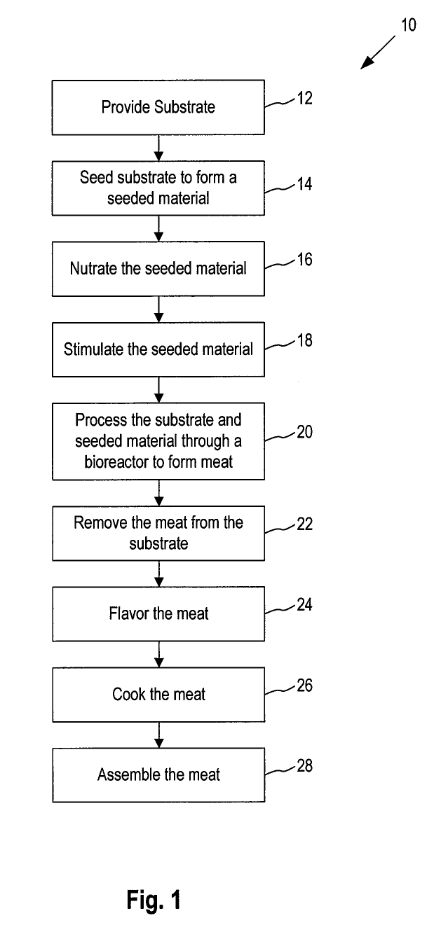

FIG. 1 is a flow diagram of an example method for producing synthetic meat in accordance with at least some examples of the present disclosure. FIG. 1 includes the following numbering to refer to operations of the example procedure, including process 10. Process 10 may include one or more functions, actions, or operations, such as is illustrated by one or more of: operation 12 (providing a substrate); operation 14 (seeding the substrate to form a seeded material); operation 16 (nutrating the seeded material); operation 18 (stimulating the seeded material); operation 20 (processing the substrate and the seeded material through a bioreactor to form meat); operation 22 (a separation step comprising removing the meat from the substrate); operation 24 (imparting flavor to the meat); operation 26 (cooking the meat); and/or operation 28 (assembling the meat). As illustrated, the example procedure may be implemented in and/or by a process 10. In some examples, various operations described herein may be divided into additional operations, combined with other operations, or eliminated as may be required in a particular example.

Further description will now be made regarding operations of the process 10 in accordance with various examples of the present disclosure. Providing a substrate 12 may comprise providing a long roll of a porous polymer to act as a support structure for cell growth. In some examples, the polymer may be 0.5 mm thick or less. The substrate may be configured to support cell growth. Seeding the substrate 14 may be done using a roll-to-roll processing method such as by a roll-to-roll mechanism. This may involve moving the substrate past a “doctor blade” where cell seeding may take place. Seeding may be done with stem cells. Seeding the substrate may provide a seeded material on a surface of the substrate. Nutrating the seeded material 16 may involve rolling the seeded substrate into a perfusion bioreactor to force nutrients and growth factors through the cells and the substrate. Stimulating the seeded material 18 may involve stretching and relaxing the substrate and/or seeded material to stimulate action of muscle cells. The amount and level of stretching and relaxing may vary based on the type of muscle or the desired tenderness. For example, cardiac tissue may stretched at approximately 80 Hz to an approximately 20% increase. Skeletal muscle may be stretched at approximately 3 Hz to an approximately 10% increase. Stretching may in some examples be done on an intermittent basis, such as for 5 minutes of every 60 minutes. In some examples, stimulating the seeded material 18 may comprise stretching the substrate at predetermined periods of time to simulate muscle action. The predetermined periods of time may generally be based on the desired tenderness of the meat. Stretching the muscle will typically make the meat more tough. Thus, where the synthetic meat is to approximate veal, little or no stretching may be done. In contrast, where the synthetic meat is to approximate chicken breast, a regimen of several stretches per hour may be done. Processing the substrate and seeded material through the bioreactor to form meat 20 may comprise feeding the substrate through the bioreactor such that the substrate exits after cell growth has formed a uniform and mechanically integral film of meat. Generally, completion of the growth process may be considered when a sheet of meat has been formed. This sheet of meat is then exited from the bioreactor. Processing may include monitoring the seeded substrate as it may be rolled through the bioreactor for uniformity of cell growth. Removing the meat from the substrate 22, or separation, may occur without intervention if the substrate degrades during processing of the substrate and seeded material through the bioreactor. Alternatively, separation may be done by processing the meat at a station designed to remove the substrate using, for example, treatment with enzymes, dilute vinegar, or lye. Generally, enzymatic treatment may be used to tenderize the meat. In some examples, the enzyme may be an enzyme mixture comprising The bromelin, ficin, papain and/or actinidin. Separation thus may comprise dissolution by treatment with acid or base, or an enzymatic cleavage process. Imparting flavor to the meat 24 may be done by moving the meat film to a station designed to impart flavor and surface texture such as by injecting flavor fluids into the meat. Flavor fluids may include an fluid that is designed to impart a specific flavor into meat. For example, suitable flavor fluids may comprise liquid smoke fluid and teriyaki fluid. Cooking the meat 26 may be done at any suitable temperature. Generally, raising the temperature of the meat to 160° F. ensures denaturing of proteins. Assembling the meat 28 may include dicing and assembling.

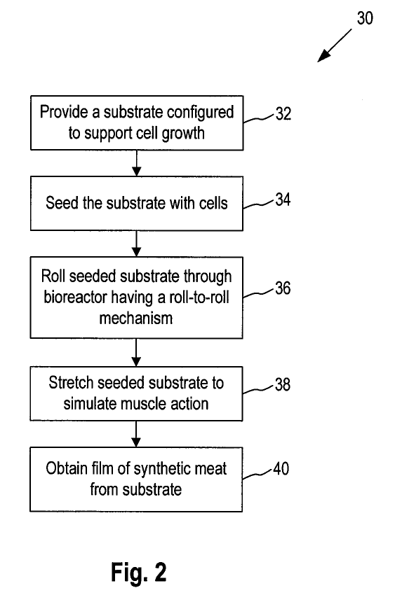

FIG. 2 is a flow diagram of an alternative example method 30 of producing synthetic meat in accordance with at least some examples of the present disclosure. Method 30 may include one or more functions, actions, or operations, such as is illustrated by one or more of: operation 32 (provide a substrate configured to support cell growth); operation 34 (seed the substrate with cells); operation 36 (roll seeded substrate through bioreactor having a roll-to-roll mechanism), operation 38 (stretch seeded substrate to simulate muscle action); and/or operation 40 (obtain file of synthetic meat from substrate). In some examples, various operations described herein may be divided into additional operations, combined with other operations, or eliminated as may be required in a particular example.

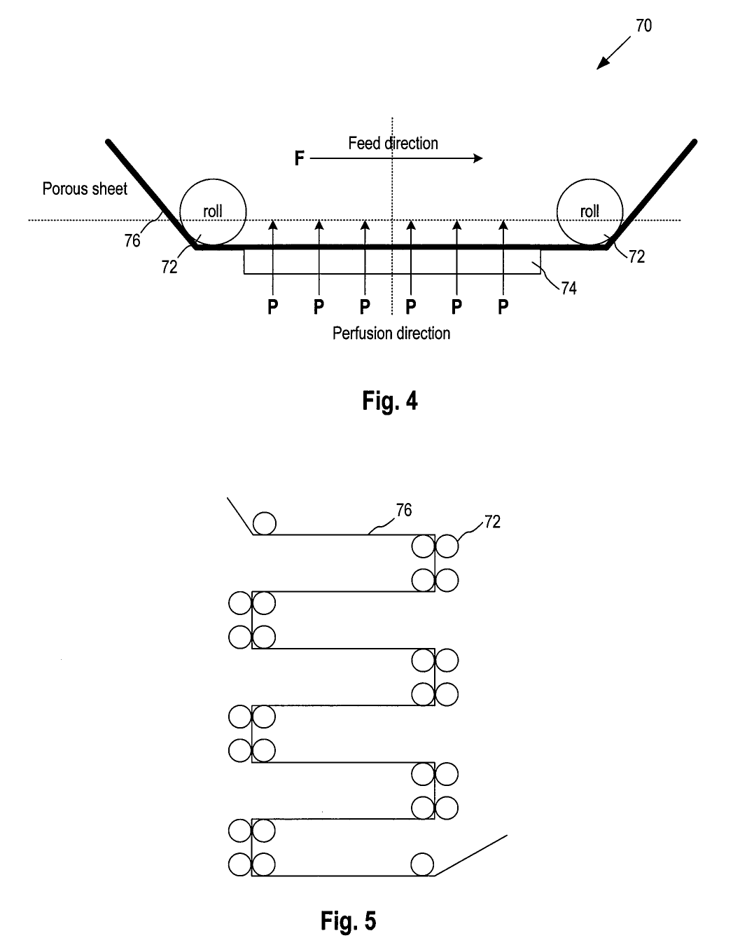

As shown, a substrate configured to support cell growth is provided at operation 32. The substrate may be seeded with cells at operation 34. The seeded substrate may be rolled through a bioreactor having a roll-to-roll mechanism at operation 36, thereby allowing nutrients and growth factors to interact with the cells. The seeded substrate may be stretched to simulate muscle action at operation 38. One example of a mechanism for providing such stretching is shown in detail in FIGS. 4 and 5, where the rolls 72 can be moved at different rates thereby elongating the sheet 76 by 5-10%. The seeded substrate may be monitored for uniformity of cell growth as it may be rolled through the bioreactor at operation 38. A film of synthetic meat may be obtained from the substrate at operation 40.

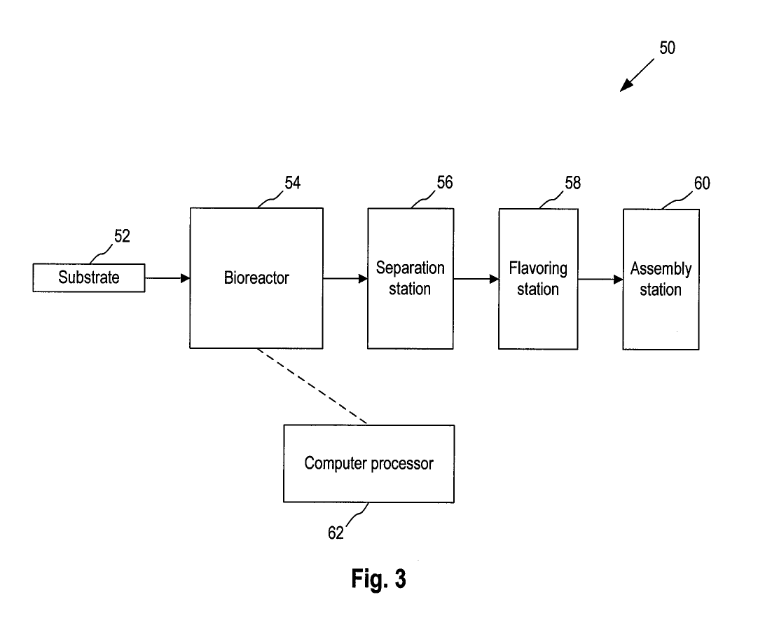

FIG. 3 is a block diagram of an example system 50 configured to be suitable for producing synthetic meat in accordance with at least some examples of the present disclosure. FIG. 3 includes numbering to designate illustrative components of examples shown within the drawing, including the system 50; a substrate 52; a bioreactor 54; a separation station 56; a flavoring station 58; an assembly station 60; and a computer processor 62. In some examples, one or more of the stations may be omitted or combined with another station. For example, the separation station 56 may be omitted or the flavoring station 58 and the assembly station 60 may be combined. These stations 58, 60 may have a plurality of tasks associated therewith. For example, at the assembly station 60, any treated synthetic meat can be cut, packaged, and/or prepared for shipping. A stretching mechanism may be included in the system, such as part of the bioreactor 54 or interacting with the substrate in the bioreactor 54, to stretch synthetic meat as it is formed.

The substrate 52 may be a porous polymer sheet such as a porous plastic sheet prepared by sintering a loose sheet of finely divided plastic powder such that the individual grains stick together but leave relatively large pore volume. Such sheets may be formed from a wide variety of plastics and at thickness of, for example 0.5 mm. In some examples, the substrate may be formed of a material that may be stretched by approximately 10% to 20% when wet in order to simulate expansion and contraction of native muscle cells during cell growth. In some examples, the substrate 52 may be formed of a polymer that degrades during processing. The substrate may be referred to as a tissue engineering scaffold.

The bioreactor may be a modified perfusion bioreactor. Perfusion bioreactors are adapted to generally mimic the conditions experienced by cells within the body. The perfusion bioreactor may be configured to produce a protein rich fluid for nutrating the seeded material. The perfusion bioreactor thus may bathe the stem cell with the protein rich fluid to provide nourishment by a parallel simulation of circulatory system. The perfusion bioreactor may be configured to allow the delivery of essential nutrients in a physiological way. The bioreactor may be modified into a roll-to-roll mechanism to process high volumes of synthetic meat, as will be described with respect to FIG. 5. While bioreactors typically may produce loose cells on denser supports, configuring a bioreactor in a roll-to-roll mechanism may facilitate production of a dense uniform layer of cells on a porous substrate.

The processor 62 may be operably associated with the bioreactor for controlling time in the bioreactor and rate of progress of the substrate through the bioreactor. Environmental conditions may be set and controlled by the processor to facilitate formation of the meat. For example, temperature may be between approximately 37° C. and 42° C., and pressure may be room pressure. The processor 62 may be associated with a monitoring system for monitoring the substrate within the bioreactor for uniformity of cell growth.

The separation station 56, flavoring station 58, and assembly station 60 may be configured as tables or other suitable design for receiving the synthetic meat and applying appropriate procedures to the synthetic meat. Separation may comprise washing and drying the meat. Flavoring may comprise coating with a flavor solution or fluid (such as previously described) or exposing to smoke. Further procedures may include dicing and packaging the meat.

FIG. 4 is a diagram of a suitable bioreactor 70 for use with systems for producing synthetic meat in accordance with at least some examples of the present disclosure. As shown, the bioreactor 70 includes first and second rolls 72 and a reservoir 74. The seeded substrate 76 may be fed through the bioreactor 70 along the rolls 72. In the example shown, a protein rich fluid, such as a solution of amino acids, may be provided in the reservoir 74 and perfusion may occur from the reservoir 74 through the substrate 76 in perfusion direction P. The substrate moves through the bioreactor in direction F. Movement through the bioreactor defines the total time of the substrate (and meat being formed) in the bioreactor. As the substrate moves through the bioreactor, the meat grows on the substrate. The rate of movement may be set to facilitate complete formation of the meat before it exits the bioreactor.

FIG. 5 is a diagram of a bioreactor 70 such as shown in FIG. 4 in a stacked configuration. In order to process high volumes of synthetic meat, the sheets of meat may be extended on rolls stacked upon each other in three dimensions, as shown in FIG. 5. FIG. 5 illustrates the rolls 72 and seeded substrate 76 as fed through the bioreactor 70.

In use, a long sheet of substrate 76 may be loaded at the beginning of a roll-to-roll line, and may be fed across multiple pieces of equipment, such as those described with respect to FIG. 3, in a continuous fashion. After seeding, cell growth may be encouraged in the perfusion bioreactor 70 of FIGS. 4 and 5. In commonly available perfusion reactors, growth media (such as the protein rich fluid) may be forced through a porous support (such as the substrate) to ensure uptake of nutrients by cells on the support. In the bioreactors of FIGS. 4 and 5, the roll of substrate may be kept pressed against construct through which oxygenated grown media flows, such as the reservoir 74.

To stimulate the extension and contraction of muscle cells, the substrate 76 may be stretched and relaxed by moving the rolls 72 shown in FIGS. 4 and 5 back and forth relative to each other. The substrate 76 may then be drawn through the bioreactor 70 at a rate such that cells are uniformly grown on the substrate 76 before exiting the bioreactor 70. Growth of the cells may be monitored and assessed by a processor for uniformity and density. The substrate 76 may be configured such that it degrades inside the bioreactor 70, or it may be configured to remain substantially intact until a subsequent separation step. Such separation step may comprise dissolution by treatment with acid or base, or an enzymatic cleavage process.

The continuous sheet of raw meat may be subsequently processed at a station that imparts flavor such as smoke and/or applies surface texture to more closely resemble the grains of real meat. The meat may then be cooked and assembled. Assembly may comprise cutting from the roller into smaller sheets such as approximating deli-meat.

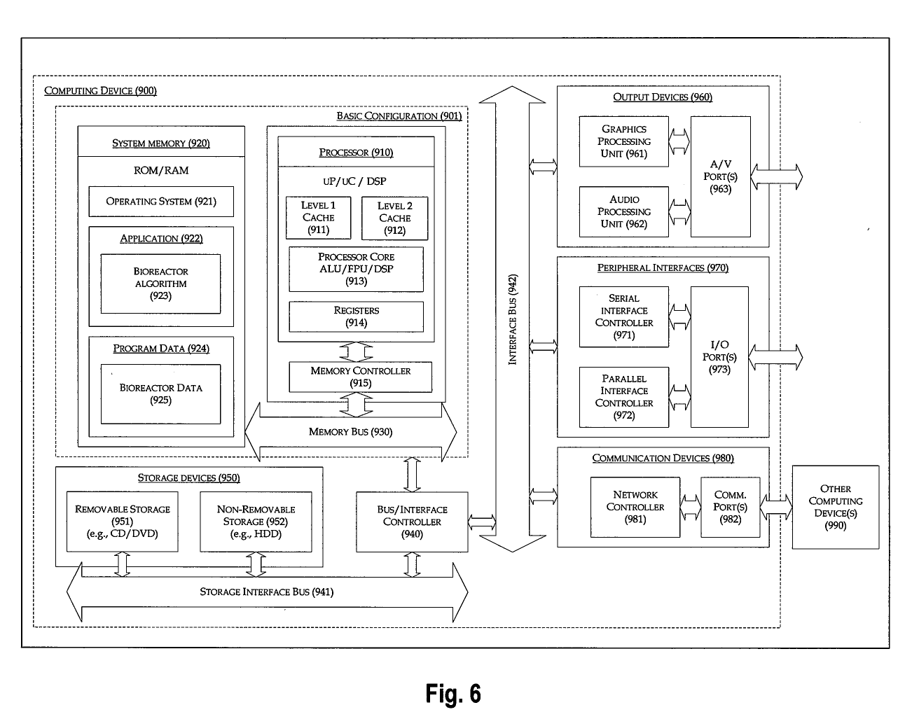

FIG. 6 is a block diagram of a computing device that may be used in producing synthetic meat according to certain examples of the present disclosure. FIG. 6 includes numbering to designate illustrative components of examples shown within the drawings, including; a computing device 900; a basic configuration 901; a processor 910; a level one cache 911; a level two cache 912; a processor core 913; registers 914; a memory controller 915; a system memory 920; an operating system 921; an application 922; a bioreactor algorithm 923; program data 924; bioreactor information 925; a memory bus 930; a bus/interface controller 940; a storage interface bus 941; an interface bus 942; a data storage device 950; a removable storage device 951; a non-removable storage device 952; an output device 960; a graphics processing unit 961; an audio processing unit 962; an A/V (audio-visual) port 963; peripheral interfaces 970; a serial interface controller 971; a parallel interface controller 972; I/O (input/output) ports 973; a communication device 980; a network controller 981; a communication port 982; and computing devices 990.

Generally, example system 600 described in relation to FIG. 3, for example, may be integrated within a computing device or environment. FIG. 6 generally illustrates a block diagram of a suitable computing device 900 by which producing synthetic meat may be implemented. In a basic configuration 901, the computing device 900 may typically include one or more processors 910 (such as processing arrangement 501) and system memory 920. A memory bus 930 may be used for communicating between the processor 910 and the system memory 920.

Depending on the desired configuration, the processor 910 may be of any type including, but not limited to, a microprocessor (μP), a microcontroller (μC), a digital signal processor (DSP), an ARM968 processor, other processor with suitable functionality and capabilities, or any combination thereof. The processor 910 may include one more levels of caching, such as a level one cache 911 and a level two cache 912, a processor core 913, and registers 914. The processor core 913 may include an arithmetic logic unit (ALU), a floating point unit (FPU), a digital signal processing core (DSP Core), or any combination thereof. A memory controller 915 may also be used with the processor 910, or in some implementations the memory controller 915 may be an internal part of the processor 910. In some examples, processor and peripherals may be integrated into a single application specific integrated circuit (ASIC).

Depending on the desired configuration, the system memory 920 may be of any type including but not limited to volatile memory (such as RAM), non-volatile memory (such as ROM, flash memory, etc.) or any combination thereof. System memory 920 may typically include an operating system 921, one or more applications 922, and program data 924. Application 922 may include a bioreactor algorithm 923 that may be arranged to develop rate, timing, and temperature instructions for the conditions at which the substrate is processed through the bioreactor. Temperature may range, for example between approximately 37° C. and approximately 42° C., or approximately body temperature for animals. Bioreactor data 925 may include, for example, bioreactor information 925, from which suitable rates may be determined. In some examples, application 922 may be arranged to operate with program data 924 on an operating system 921 such that synthetic meat may be processed in accordance with the methods and processes describer herein. This described basic example configuration is illustrated in FIG. 6 by those components within dashed line 901.

The computing device 900 may have additional features or functionality, and additional interfaces to facilitate communications between the example basic configuration 901 and any devices and interfaces. For example, a bus/interface controller 940 may be used to facilitate communications between the example basic configuration 901 and one or more data storage devices 950 via a storage interface bus 941. The data storage devices 950 may be removable storage devices 951, non-removable storage devices 952, or a combination thereof. Examples of removable storage and non-removable storage devices include, e.g., magnetic disk devices such as flexible disk drives and hard-disk drives (HDD), optical disk drives such as compact disk (CD) drives or digital versatile disk (DVD) drives, solid state drives (SSD), and tape drives. Example computer storage media may include volatile and nonvolatile, removable and non-removable media implemented in any method or technology for storage of information, such as, e.g., computer readable instructions, data structures, program modules, or other data.

System memory 920, removable storage 951 and non-removable storage 952 are examples of computer storage media. Computer storage media may include, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 900. Any such computer storage media may be part of device 900.

The computing device 900 may also include an interface bus 942 for facilitating communication from various interface devices (e.g., output interfaces, peripheral interfaces, and communication interfaces) to the basic configuration 901 via the bus/interface controller 940. Example output devices 960 may include a graphics processing unit 961 and an audio processing unit 962, which may be configured to communicate to various external devices such as a display or speakers via one or more A/V ports 963. Example peripheral interfaces 970 include a serial interface controller 971 or a parallel interface controller 972, which may be configured to communicate with external devices such as input devices (e.g., keyboard, mouse, pen, voice input device, touch input device, etc.) or other peripheral devices (e.g., printer, scanner, etc.) via one or more I/O ports 973.

An example communication device 980 may include a network controller 981, which may be arranged to facilitate communications with one or more other computing devices 990 over a network communication via one or more communication ports 982. The communication connection is one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), infrared (IR) and other wireless media. The term computer readable media and/or computer-accessible medium as used herein may include, e.g., both storage media and communication media, for example.

The computing device 900 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. The computing device 900 may also be implemented as a personal computer including both laptop computer and non-laptop computer configurations. The computing device 900 may also be implemented as an interactive system such as an information kiosk, television, or a gaming device.

Provided and described herein are example implementations of a computer-accessible medium containing executable instructions thereon, wherein a processor such as provided in computing device 900 may be adapted to execute instructions such that the processor may be configured to receive information related to formed meat on a substrate in a bioreactor, determine a rate for processing the substrate through the bioreactor, and outputting the meat from the bioreactor.

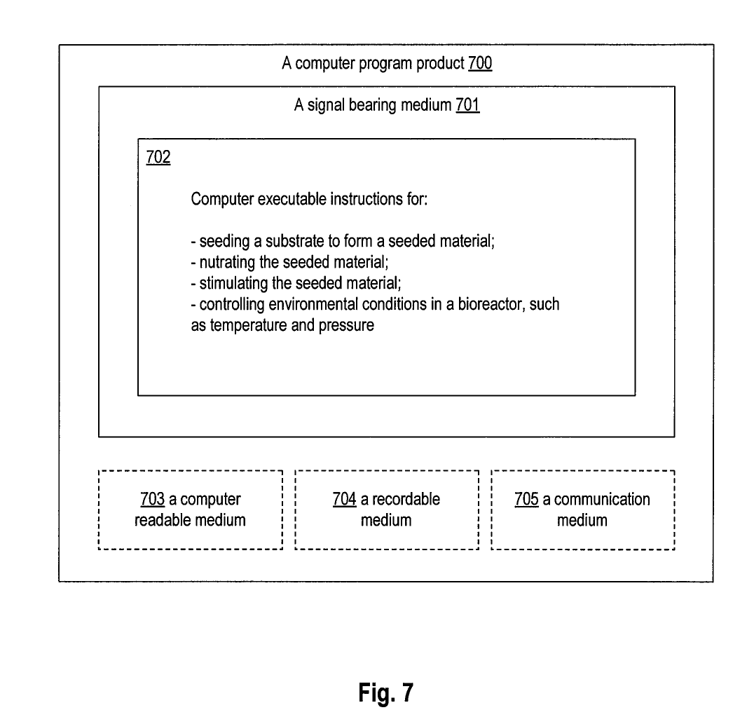

FIG. 7 illustrates a block diagram of an example computer program product in accordance with the present disclosure. In some examples, a computer program product 700 includes a signal bearing medium 701 that may also include computer executable instructions 702. Computer executable instructions 702 may be arranged to provide instructions for forming synthetic meat. Such instructions may include, for example, instructions relating to seeding a substrate to form a seeded material. Such instructions further may include, for example, instructions relating to nutrating the seeded material. Such instructions further may include, for example, instructions relating to stimulating the seeded material. Such instructions further ay include, for example, instructions related to controlling environmental conditions in a bioreactor, such as temperature and pressure. Generally, the computer executable instructions 702 may include instructions for performing any steps of the method for forming synthetic meat described herein.

Also depicted in FIG. 7, in some examples, computer product 700 may include one or more of a computer readable medium 703, a recordable medium 704 and a communications medium 705. The dotted boxes around these elements may depict different types of mediums that may be included within, but not limited to, signal bearing medium 701. These types of mediums may distribute programming instructions 702 to be executed by computer devices including processors, logic and/or other facility for executing such instructions. Computer readable medium 703 and recordable medium 704 may include, but are not limited to, a flexible disk, a hard disk drive (HDD), a Compact Disc (CD), a Digital Video Disk (DVD), a digital tape, a computer memory, etc. Communication medium 705 may include, but is not limited to, a digital and/or an analog communication medium (e.g., a fiber optic cable, a waveguide, a wired communication link, a wireless communication link, etc.).

Provided and described herein, for example, are example implementations of various methods for producing synthetic meat. Example methods may seeding a porous substrate with seed cells and feeding the seeded substrate through a perfusion bioreactor using roll-to-roll methodology. The methods thus describe a continuous process for the growth of synthetic meat, which may be used in large-scale implementation. In specific examples, the methods described herein may produce meat having a thickness of 0.5 mm or below, such as suitable for deli meats. Such meat does not require vascularization.

Also provided and described herein are examples of systems arranged in accordance with the present disclosure for producing synthetic meat. Example systems may comprise a perfusion bioreactor configures as a roll-to-roll mechanism and one or more processing stations. Example systems may also comprise a processor operably associated with the bioreactor and configured to monitor and assess uniformity and density of meat formed on a substrate.

The present disclosure is not to be limited in terms of the particular examples described in this application, which are intended as illustrations of various aspects. Many modifications and variations may be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which may, of course, vary. It is also to be understood that the terminology used herein is for the purpose of describing particular examples only, and is not intended to be limiting.

There is little distinction left between hardware and software implementations of aspects of systems; the use of hardware or software is generally (but not always, in that in certain contexts the choice between hardware and software may become significant) a design choice representing cost vs. efficiency tradeoffs. There are various vehicles by which processes and/or systems and/or other technologies described herein may be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various examples of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. In one example, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the examples disclosed herein, in whole or in part, may be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative example of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art may translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub ranges and combinations of sub ranges thereof. Any listed range may be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like include the number recited and refer to ranges which may be subsequently broken down into sub ranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and examples have been disclosed herein, other aspects and examples will be apparent to those skilled in the art. The various aspects and examples disclosed herein are for purposes of illustration and are not intended to be limiting, with the true scope and spirit being indicated by the following claims.

What is claimed is:

1. A method for producing synthetic meat on an industrial scale, comprising:
configuring a substrate to support cell growth;
seeding the substrate with cells;
rolling the seeded substrate into a bioreactor having a roll-to-roll mechanism thereby allowing nutrients and growth factors to interact with the cells;
stimulating the seeded substrate in the bioreactor to simulate muscle action;
monitoring the seeded substrate as it is being rolled in the bioreactor for uniformity of cell growth; and
obtaining a film of synthetic meat from the substrate.

2. The method of claim 1, further comprising providing nutrients to the seeded substrate.

3. The method of claim 1, wherein the substrate comprises a porous polymer substrate.

4. The method of claim 1, wherein stimulating the seeded substrate comprises stretching the seeded substrate.

5. The method of claim 4, wherein the stretching comprises stretching at predetermined periods of time to simulate muscle action.

6. The method of claim 1, further comprising adding a texture to the film of synthetic meat.

7. The method of claim 1, further comprising adding a flavor to the film of synthetic meat.

8. The method of claim 1, further comprising separating the film of synthetic meat from the substrate.

9. The method of claim 8, wherein the separating occurs while rolling the seeded substrate into the bioreactor by disintegration of the substrate.

10. The method of claim 8, wherein the separating comprises dissolution by acid or base.

11. The method of claim 8, wherein the separating comprises using an enzymatic cleavage process.

12. The method of claim 1, further comprising assembling the synthetic meat.

13. The method of claim 1 wherein the rolling of the seeded substrate through the bioreactor comprises nutrating the seeded substrate.

14. The method of claim 1, further comprising cooking the synthetic meat.

15. A system for producing synthetic meat, comprising:
a roll-to-roll perfusion bioreactor configured to receive a seeded substrate and apply a fluid to the seeded substrate such that the seeded substrate forms a thin film of synthetic meat;
a flavoring station configured to treat the synthetic meat received from the roll-to-roll perfusion bioreactor;
an assembly station configured to receive the treated synthetic meat from the flavoring station, wherein the treated synthetic meat is at least one of cut, packaged, or prepared for shipping at the assembly station; and
a computer operably associated with the bioreactor and configured to assess uniformity of cell growth on the seeded substrate and configured to control a rate of progress of the seeded substrate through the bioreactor based at least on the assessed uniformity of cell growth.

16. The system of claim 15, wherein the computer is configured to control temperature in the bioreactor.

17. The system of claim 15, further comprising a substrate.

18. The system of claim 17, wherein the substrate comprises a porous polymer sheet.

19. The system of claim 15, wherein the flavoring station is configured to impart one or more of flavor and/or texture to the synthetic meat.

20. The system of claim 15, further comprising a separation station configured to receive the synthetic meat from the bioreactor and separate the synthetic meat from the substrate.

21. The system of claim 15, further comprising a monitor configured to monitor thin film growth on the seeded substrate, wherein the monitor is adapted to communicate with the processor.

22. The system of claim 15, further comprising a mechanism for stretching the seeded substrate using a set of rolls.

23. A computer-accessible medium containing executable instructions thereon, wherein when a processor executes the instructions, the processor is configured to:
roll a seeded substrate into a bioreactor having a roll-to-roll mechanism, thereby allowing nutrients and growth factors to interact with the cells;
stretch the seeded substrate to simulate muscle action;
monitor the seeded substrate as it is being rolled in the bioreactor for uniformity of cell growth; and
obtain a film of synthetic meat from the substrate.

24. The computer-accessible medium of claim 23 wherein the processor is configured to execute instructions relating to rate of progress of the seeded substrate through the bioreactor.
